(19) United States
(12) Patent Application Publication
(10) Pub. No.: US 2010/0106181 A1
(43) Pub. Date: Apr. 29, 2010

(54) IN-SITU FILTER

(76) Inventors: Yossi Gross, Moshav Mazor (IL); Daniel Goldstein, Efrat (IL)

Correspondence Address: SUGHRUE MION, PLLC, 2100 PENNSYLVANIA AVENUE, N.W., SUITE 800, WASHINGTON, DC, 20037, US

(21) Appl. No.: 12/522,467

(22) PCT Filed: Jan. 8, 2008
(86) PCT No.: PCT/IL08/00038

§ 371 Date: Oct. 7, 2009

Related U.S. Application Data
(60) Provisional application No. 60/879,391, filed on Jan. 8, 2007.

(51) Int. Cl. A61F 2/01

(52) U.S. Cl. 606/200

(57) ABSTRACT

Apparatus is provided including a filter (30) configured for placement into a body of a patient, in a vicinity of a site including cancerous tissue (28). The filter (30) comprises an attachment surface (34) configured to capture particles (26) administered to treat the tissue (28). Other embodiments are also described.

### CROSS-REFERENCES TO RELATED APPLICATIONS

The present application claims priority from U.S. Provisional Patent Application 60/879,391 to Gross, filed Jan. 8, 2007, entitled, “In-situ chemotherapy filter,” which is incorporated herein by reference.

### FIELD OF THE INVENTION

The present invention generally relates to implantable medical apparatus. Specifically, the present invention relates to implantable apparatus to reduce toxicity associated with chemotherapy, radiotherapy, and contrast agents.

### BACKGROUND OF THE INVENTION

Cancer, today, remains among the leading cause of premature death around the world. Varying forms of treatment have developed over the last few decades; among them: lumpectomy, removal of the axillary lymph nodes to ascertain tumor progression, radiation therapy, hormone therapy, monoclonal antibodies and chemotherapy. Chemotherapy is a treatment used for some types of cancer and can be applied, at lower doses, to treat non-cancerous conditions, as well. However, non-specific targeting, as well as excess dosage of chemotherapeutic agents pose a toxicity threat to, and occasionally, compromise the viability of healthy tissue.

Radiopaque dyes are often used to facilitate imaging procedures, and are also sometimes associated with a toxicity threat to healthy tissue of a subject.

As described by Wikipedia, photodynamic therapy (PDT) is a treatment for cancer, acne, wet macular degeneration, and other conditions. A photosensitizer is a chemical compound that can be excited by light of a specific wavelength, e.g., visible or near-infrared light. In photodynamic therapy, either a photosensitizer or the metabolic precursor of one is administered to a patient. The tissue to be treated is exposed to light suitable for exciting the photosensitizer.

U.S. Pat. No. 5,069,662 to Bodden, et al., which is incorporated herein by reference, describes perfusing a high concentration of an agent to treat an organ, such as anti-cancer agents through a body organ containing a tumor, without their entering the body's general circulation; removing them from the organ with effluent blood; and transporting the contaminated blood to an extracorporeal circuit where the blood is treated to remove the contamination, and returning the treated blood to the body. The process is described as preventing toxic levels of the agents from entering the body's general circulation while delivering lethal doses of the agents to the tumor. There are described various apparatus for effecting intra- and extracorporeal treatment of such contaminated blood.

U.S. Pat. No. 6,186,146 to Glickman, et al., which is incorporated herein by reference, describes an in situ treatment of a cancerous organ. The method includes: subjecting a diseased or tumorous organ to an effective amount of a therapeutic agent by infusing the agent via blood entering the organ; creating an isolated section in a major vein spanning the area where the tributary veins connect with the major vein, the major vein and tributary veins being directly associated with the organ; passing contaminated effluent blood from the tributary veins of the organ to the isolated section and capturing the effluent blood therein; and, evacuating the captured blood from the isolated section without exposing the contaminated effluent blood to other organs or tissues of the body and without interrupting the general circulation in the system of the body.

U.S. Pat. No. 5,427,767 to Kresse, et al., which is incorporated herein by reference, describes nanocrystalline magnetic particles consisting of magnetic iron oxide core as well as the use thereof in medical diagnostics and/or therapy. The magnetic particles are characterized by composition of the coating material of natural or synthetic glycosaminoglycans and/or their derivatives with molecular weights of 500 Da to 250,000 Da, if necessary, covalently cross-linked with appropriate cross-linking agents and/or modified by specific additives. The medical diagnostic usefulness of coated ferric/ferromagnetic (superparamagnetic) iron oxide particles (parent substance) is based on the fact that, following intravenous injection, they are taken up by phagocytizing monocytes and macrophages of the reticuloendothelial system (RES) of the clinically intact splenic and hepatic tissue but are not taken up by tumors and metastases.

U.S. Pat. No. 4,735,796 to Gordon, et al., which is incorporated herein be reference, discusses post-treatment practice, and gives consideration to the removal of ferromagnetic, paramagnetic or diamagnetic particles from the subject. The removal is accomplished by natural excretory processes which may be supplemented with chelating agents or metal efflux stimulating compositions.

US Patent Application Publication 2006-0142749 to Ivkov et al., which is incorporated herein by reference, describes thermotherapeutic compositions for treating disease material, and methods of targeted therapy utilizing such compositions. These compositions comprise a) stable single domain magnetic particles; b) magnetic nanoparticles comprising aggregates of superparamagnetic grains; or c) magnetic nanoparticles comprising aggregates of stable single magnetic domain crystals and superparamagnetic grains. These compositions may also comprise a radio isotope, potential radioactive isotope, chemotherapeutic agent. These methods comprise the administration to a patient's body, body part, body fluid, or tissue of bioprobes (energy susceptive materials attached to a target-specific ligand), and the application of energy to the bioprobes so as to destroy, rupture, or inactivate the target in the patient. Energy forms, such as AMF, are utilized to provide the energy. The disclosed methods are described as being useful in the treatment of a variety of indications, including cancers, diseases of the immune system, central nervous system and vascular system, and pathogen-borne diseases.

U.S. Pat. No. 4,323,056 to Borrelli, et al., which is incorporated herein by reference, describes a noninvasive tumor treatment modality which is described as resulting in a reduction of tumor mass and possibly leading to complete eradication of a tumor. The method comprises localized magnetically-coupled, RF-induced hyperthermia mediated by a material which is non-toxic to and, preferably, compatible with animal tissue and has incorporated therewithin iron-containing crystals of such size, amount, composition, and magnetic properties to impart a coercive force of at least 200 oersteds to the material, and wherein the RF magnetic field has a frequency not in excess of about 10 kilohertz.

U.S. Pat. No. 6,514,481 to Prasad et al., which is incorporated herein by reference, describes nanosized particles termed as “nanoclinics” for therapeutic and/or diagnostic use. The particles have a core made of a therapeutic or diagnostic material surrounded by a shell. Further, the particles contain a targeting agent on the surface of the shell for specific recognition of targeted cells. A method is also described for lysis of cells using a DC magnetic field. Further, a method is described for fabrication of nanoclinics that can target and lyse specific cells such as cancer cells.

U.S. Pat. No. 6,530,944 to West et al., which is incorporated herein by reference, describes methods for the localized delivery of heat and the localized imaging of biological materials. The delivery may be in vitro or in vivo and is useful for the localized treatment of cancer, inflammation or other disorders involving overproliferation of tissue. The method is also useful for diagnostic imaging. The method involves localized induction of hyperthermia in a cell or tissue by delivering nanoparticles to said cell or tissue and exposing the nanoparticles to an excitation source under conditions wherein they emit heat.

PCT Publication WO 06/108405 to Jordan et al., which is incorporated herein by reference, describes nanoparticles, whereby at least one therapeutically active substance is bonded to the nanoparticles and the release of the therapeutically active ingredient is brought about or initiated by an alternating magnetic field. The publication further relates to pharmaceutical compositions, in particular, injection solutions comprising said nanoparticle and the use thereof for the treatment of cancer.

The following patents and patent applications, which are incorporated herein by reference, may be of interest:
PCT Publication WO 04/068405 to Henrichs et al.
PCT Publication WO 05/76729 to Sela
U.S. Pat. No. 6,565,887 to Gray et al.
U.S. Pat. No. 6,953,438 to Milo
U.S. Pat. No. 6,599,234 to Gray et al.
U.S. Pat. No. 6,997,863 to Handy et al.
U.S. Pat. No. 7,074,175 to Handy et al.
US Patent Application Publication 2006/0058853 to Bentwich
US Patent Application Publication US 2007/0260144 to Sela

An article by Ravikumar T S et al., entitled, “Percutaneous hepatic vein isolation and high-dose hepatic arterial infusion chemotherapy for unresectable liver tumors,” Journal of Clinical Oncology, 1994; 12:2723-2736, which is incorporated herein by reference, describes a percutaneous isolated chemotherapy perfusion approach for treating advanced primary and metastatic liver tumors. Chemotherapy was administered via a hepatic artery catheter, and hepatic venous blood isolated by a percutaneous double-balloon inferior vena cava (IVC) catheter was passed through a detoxification/filtration cartridge in a venovenous bypass circuit. The use of a double-balloon catheter to isolate and detoxify hepatic venous blood during intraarterial therapy is described as being technically feasible and safe, and allows administration of large doses of intrahepatic chemotherapy at short intervals. This approach is described as allowing new dose-intensification strategies to increase tumor responses in primary and metastatic liver tumors.

The following articles, which are incorporated herein by reference, may be of interest:
Pingpank J F et al., “Phase I Study of Hepatic Arterial Melphalan Infusion and Hepatic Venous Hemofiltration Using Percutaneously Placed Catheters in Patients With Unresectable Hepatic Malignancies,” Journal of Clinical Oncology 23(15):3465-3474 (2005)
Savier E et al., “Percutaneous Isolated Hepatic Perfusion for Chemotherapy: A Phase 1 Study” Archives of Surgery 138(3):325-332 (2003)
Czauderna P et al., “Hepatocellular Carcinoma in Children: Results of the First Prospective Study of the International Society of Pediatric Oncology Group” Journal of Clinical Oncology 20(12):2798-2804 (2002)
Kusunoki N et al., “Effect of Sodium Thiosulfate on Cisplatin Removal With Complete Hepatic Venous Isolation and Extracorporeal Charcoal Hemoperfusion: A Pharmacokinetic Evaluation” Annals of Surgical Oncology 8(5):449-457 (2001)
Alexander Jr H R et al., “Current Status of Isolated Hepatic Perfusion With or Without Tumor Necrosis Factor for the Treatment of Unresectable Cancers Confined to Liver” Oncologist 5(5):416-424 (2000)
Kishi K et al., “T1 and T2 Lip Cancer: A Superselective Method of Facial Arterial Infusion Therapy-Preliminary Experience” Radiology 213(1):173-179 (1999)
Virginia Commonwealth University et al., “Magnetic nanoparticles for potential cancer treatment,” Nanotechnology (2005)
Chan et al., “Synthesis and evaluation of colloidal magnetic iron oxides for the site-specific radiofrequency-induced hyperthermia of cancer,” Journal of Magnetism and Magnetic Materials 122 374-378 (1993)

### SUMMARY OF THE INVENTION

In some embodiments of the present invention, a filter housing comprising a filter comprising one or more attachment surfaces is designated for insertion into the vasculature of a patient diagnosed with cancer. The filter housing is typically placed at a venous site, downstream of cancerous tissue, and is configured such that the attachment surfaces are capable of capturing, intracorporeally, potentially toxic particles configured to treat cancerous tissue (e.g., chemotherapeutic particles and/or radiotherapeutic particles) and/or contrast agent particles (e.g., ultrasound contrast agent particles and/or a radiopaque dye particles), escaping the target area to which they have been administered. Typically, the particles comprise nanoparticles. Typically, the contrast agent particles are used during a diagnostic procedure and/or during treatment in order to locate a position of the cancerous tissue.

For some applications, placing the filter downstream of the cancerous tissue restricts blood flow away from the cancerous tissue, thereby increasing blood pressure within vasculature supplying the cancerous tissue, consequently enhancing diffusion of the chemotherapeutic particles, radiotherapeutic particles, ultrasound contrast agent particles and/or radiopaque dye particles into the cancerous tissue.

Alternatively or additionally, placing the filter upstream of the cancerous tissue restricts circulation to the cancerous tissue, inhibiting activity or effecting hypoxia-induced cell death in the cancerous tissue.

In an embodiment, in addition to or instead of placement of the filter downstream of the site containing cancerous tissue, a filter is placed at a site within the vasculature of the patient upstream of non-cancerous tissue, in order to reduce the incidence of toxic, excess treatment particles and/or contrast agent particles that have escaped the target area, from affecting the non-cancerous tissue.

The attachment surfaces of the filter typically comprise means for attracting the treatment particles and/or contrast agent particles. For example, a magnet may be used to attract magnetic particles bound to the treatment particles and/or contrast agent particles. Alternatively, antibodies functioning as receptors possessing affinity to ligands associated with the treatment particles and/or contrast agent particles are used to attract the treatment particles and/or contrast agent particles.

In some embodiments, a protein is coupled to each particle, and the antibodies coupled to the attachment surfaces are configured to bind to the protein coupled to the particle. In some embodiments, the particle remains bound to the attachment surface of the filter via the bond between the antibody and the protein coupled to the particle. Alternatively or additionally, the antibody is configured to neutralize and/or detoxify the particle bound thereto either directly or indirectly (i.e., via the protein coupled to the particle). In such an embodiment, once directly or indirectly bound to the particle, the antibody undergoes a conformational change in order to physically reduce the effectiveness of the particle. In either embodiment, following the detoxification of the particle, the particle either remains coupled to the filter and/or is allowed to migrate therefrom.

In some embodiments, the attachment surfaces are coupled to enzymes whose active sites target and detoxify the particles. For embodiments in which chemotherapy particles are administered to the patient, the enzyme comprises an enzyme (e.g., aldehyde dehydrogenase or glutathion-S-transferase) that metabolizes and detoxifies chemicals of the chemotherapy particle.

In some embodiments of the present invention, the attachment surfaces of the filter are charged in response to an application of voltage thereto. In some embodiments, the voltage is applied to the surfaces using an electrode implanted within the body of the patient adjacently to the filter. In such an embodiment, the particles comprise charged nanoparticles which are attracted to the charged attachment surfaces of the filter. The charged nanoparticles are typically insulated by and enveloped within nanocontainers, e.g., buckyballs, when administered to the patient.

For some applications, the one or more attachment surfaces comprise a single attachment surface, typically configured to have a large surface area within the housing. Alternatively, a plurality of attachment surfaces are disposed with respect to the housing of the filter such that a first one of the surfaces captures a first subset of the treatment and/or contrast agent particles within the bloodstream flowing therethrough, and a second one of the surfaces captures a second subset of the particles.

In some embodiments of the present invention, a flow restricting element, typically but not necessarily shaped to define an internal channel, is designated for insertion into the vasculature of a patient diagnosed with cancer. In an embodiment, the flow restricting element is placed at an arterial site, upstream of cancerous tissue, or at a venous site, downstream of the cancerous tissue, in order to reduce blood circulation to the cancerous tissue, consequently inhibiting activity or effecting hypoxia-induced cell death in the cancerous tissue.

For some applications, as a supplement to administering the treatment and/or contrast agent particles, the flow restricting element is designated for insertion into the vasculature of the patient, at a site downstream of the cancerous tissue. In this case, blood circulation away from the cancerous tissue is restricted, resulting in increased blood pressure within vasculature supplying the cancerous tissue, thereby enhancing diffusion of the particles into the cancerous tissue. The flow restricting element is designated to be placed at a site that provides venous return for the cancerous tissue.

Alternatively or additionally, the flow restricting element is designated to be placed at an arterial site, upstream of non-cancerous tissue, or at a venous site, downstream of non-cancerous tissue, thereby reducing the number of treatment and/or contrast agent particles flowing through the non-cancerous tissue.

In an embodiment, the filter and/or flow restricting element are configured to dwell transiently within the vasculature of the patient, e.g., for a period of less than one month. In this case, multiple cancer-treatment procedures using treatment particles and/or contrast agent particles are typically performed over the time that the filter and/or flow restricting element are disposed within the patient. Subsequently (e.g., at the end of chemotherapy), the filter and/or the flow restricting element are removed. Alternatively, the removal of the filter and/or flow restricting element occurs shortly following each cancer treatment procedure, and a new filter and/or flow restricting element are placed in the patient's body shortly before the following treatment procedure. In this case, the filter and/or flow restricting element are typically coupled to a catheter, to facilitate removal following the cancer treatment procedure.

In an embodiment, the particles used in the cancer-treatment procedure are coupled to a material that is sensitive to electromagnetic radiation. For example, the material coupled to the particle may be sensitive to visible light. In such an embodiment, a light source coupled to the filter is configured to affect the light-sensitive material and thereby detoxify the particle coupled thereto. Alternatively or additionally, the light source is not physically coupled to the filter, e.g., the light source is disposed adjacently to the filter or the light source is disposed externally to the body of the patient. In some embodiments, the light source is used independently of the filter. As appropriate, techniques known in the art for irradiating a material, e.g., a molecule, to cause a change of conformation thereof or to destroy the material may be utilized in combination with corresponding embodiments described herein. For some applications, techniques known in photodynamic therapy are adapted for use with embodiments of the present invention, mutatis mutandis.

Alternatively or additionally, the material coupled to the particle is sensitive to infrared radiation, and application of infrared radiation to the material detoxifies the particle. In such an embodiment, an energy transducer is configured to transmit infrared radiation toward the material coupled to the particle. In some embodiments, the energy transducer is coupled to the filter. Alternatively or additionally, the energy transducer is disposed remotely from the filter, e.g., externally to the body of the patient. In some embodiments, the energy transducer is used independently of the filter.

In some embodiments, the particles, e.g., nanoparticles, are synthesized such that the particles are susceptible at least a portion thereof to energy applied thereto. When energy is transmitted toward the particle, the particles are detoxified because the energy weakens their construction.

In some embodiments, the particles, e.g., nanoparticles, are synthesized such that the particles are susceptible at least a portion thereof to chemicals applied thereto. When the chemicals are applied to the particles, the particles are detoxified because the applied chemicals interfere with chemical bonds of the particle.

In some embodiments, the particles are coupled to a material that is positively responsive to transmitted energy (e.g., ultrasound energy, radiofrequency energy, and/or another form of electromagnetic energy). In some embodiments, the energy is transmitted from a source external to the body of the patient. Alternatively or additionally, the energy is transmitted from a source within the body of the patient, e.g., from a transducer coupled to the filter. In response to the energy applied to the material, the particle is deflected toward a vicinity of choice, e.g., a surface or trap of the filter. For example, each particle may be coupled to a material sized and/or shaped to be responsive to ultrasound energy. In response to ultrasound energy transmitted from an ultrasound transducer, the particles are deflected toward the surface or trap of the filter. In an embodiment, techniques used by Neurosonix (Rehovot, Israel) and/or described in references in the Background section of the present patent application are utilized to provide the ultrasound-based deflection.

Alternatively or additionally, the particles are coupled to and/or comprise magnetic and/or metallic particles. In response to a magnetic field applied within or to the body of the patient, the particles are deflected toward the filter. In some embodiments, the particles comprise radiotherapeutic nanoparticles which are synthesized such that they generate magnetic fields which attract them to the filter. Alternatively, these particles are synthesized such that they do not generate magnetic fields, but respond to magnetic fields applied thereto.

For some applications, each particle is coupled to a material that is responsive to electromagnetic energy, e.g., ultraviolet energy or infrared energy, and is detoxified in response to the applied energy. For some applications, each particle is coupled to a material which is responsive to electrical energy. In such an embodiment, electrodes are positioned in communication with the body of the patient and are used to deflect the particles within the vasculature of the patient via the materials coupled to the particles.

In like manner, applying energy toward the materials positively responsive to transmitted energy facilitates deflection of the particles away from tissue not designated for treatment and/or diagnosis. For example, chemotherapeutic nanoparticles used to treat cancerous tissue may be coupled to the abovementioned materials. In response to energy transmitted toward the material, the chemotherapeutic nanoparticles are deflected away from non-cancerous tissue.

In some embodiments, the light source is disposed at a distal end of a catheter which is introduced downstream of the cancerous tissue. The light source coupled to the catheter may be used independently of or in combination with the filters described herein.

In an embodiment, the particles used in the cancer treatment procedure are coupled to a material that is sensitive to radiofrequency energy or ultrasound energy. In embodiments in which the material is sensitive to ultrasound energy, the filter is coupled to an ultrasound transducer which is configured to receive ultrasound transmitted from a site outside the body of the patient. The ultrasound energy typically destroys the ultrasound-sensitive material and thereby detoxifies the particle coupled thereto. In some embodiments, the particles are coupled to a heat-sensitive material. In response to the transmitted ultrasound, localized heat is generated in the vicinity of the cancer tissue. The localized heat is sufficient to destroy the heat-sensitive material and thereby detoxify the particle coupled thereto.

In some embodiments, the ultrasound transducer is disposed at a distal end of a catheter which is introduced downstream of the cancerous tissue. The transducer is configured to transmit ultrasound energy to the cancerous tissue. Alternatively, the transducer is configured to receive energy from a transmitter disposed outside the body of the patient. The ultrasound transducer coupled to the catheter may be used independently of or in combination with the filters described herein. Alternatively or additionally, the techniques described with respect to ultrasound are practiced using radiofrequency energy, instead.

In an embodiment, antibodies to the cancerous tissue are coupled to materials, e.g., gold, that are configured to generate hyperthermic conditions in the cancerous tissue in response to applied stimulation. Typically, the antibodies are administered to the patient and bind to the cancerous tissue. Subsequently, a source of radiation, e.g., a transmitter disposed outside the body of the patient, is configured to transmit to the vicinity of the cancerous tissue sufficient radiation in order to heat the materials bound to the antibodies. In response to the heating of the materials, hyperthermic conditions are generated in the cancerous tissue, which destroy the cancerous tissue at least in part. Antibodies that did not bind to the cancerous tissue are removed from the blood stream by the filter. In an embodiment, the attachment surfaces of the filter are coupled to antibodies to at least a portion of the antibodies bound to the hyperthermia-inducing materials. For embodiments in which the material includes a metal (e.g., gold), the attachment surfaces of the filter are coupled to chelators which attract the hyperthermia-inducing materials.

The antibodies coupled to the hyperthermia-inducing materials may be administered to the patient in combination with or independently of the cancer-treatment particles.

Techniques described hereinabove may be used in combination with a medical procedures in which a contrast agent, e.g., a radiopaque dye or an ultrasound contrast agent, is administered. For example, a radiopaque dye may be administered to the patient during a diagnostic procedure, e.g., a procedure for locating a position of an aneurysm or a procedure for locating a position of a thrombosis. In such an embodiment, a filter is positioned downstream of the aneurysm in order to remove the radiopaque dye from the bloodstream of the patient. Alternatively or additionally, the filter is placed upstream of an organ so as to restrict passage of the radiopaque dye into a vicinity of the organ. In another embodiment, a radiopaque dye is administered during a therapeutic procedure, e.g., implantation of a coronary stent, and a filter is positioned to remove the dye from the bloodstream and reduce any toxicity associated with the dye.

There is therefore provided, in accordance with an embodiment of the present invention, apparatus including:

a filter configured for placement into a body of a patient, in a vicinity of a site including cancerous tissue, the filter including an attachment surface configured to capture particles administered to treat the tissue.

In an embodiment, the particles include nanoparticles.

In an embodiment, the particles include a chemotherapeutic agent coupled to a ligand, and the attachment surface includes receptors possessing affinity to the ligand.

In an embodiment, the filter is configured to dwell in the patient for a period of less than one month.

In an embodiment, the filter includes a housing, and the attachment surface includes a plurality of attachment surfaces, coupled at respective sites to the housing.

In an embodiment, the attachment surface is coupled to at least one enzyme configured to metabolize the particles.

In an embodiment, the apparatus includes a magnet in communication with the body of the patient, each particle is coupled to a material responsive to a magnetic field emitted from the magnet, and the particle is directed toward a vicinity within the body of the patient in response to the magnetic field.

In an embodiment, the attachment surface is configured to be electrically charged by a first charge, and each particle is electrically charged by a second charge, the first charge configured to attract the second charge.

In an embodiment, the apparatus includes a nanocontainer configured to insulate the particles.

In an embodiment, the apparatus includes at least one electrode in communication with the attachment surface, the at least one electrode configured to apply the first charge to the attachment surface.

In an embodiment, the attachment surface includes at least one type of antibody.

In an embodiment, the antibody is configured to detoxify the particles by neutralizing an active site of the particle.

In an embodiment, the antibody is configured to:

reversibly couple each particle,

detoxify the particle, and

release the particle subsequently to the detoxification of the particle.

In an embodiment, the attachment surface includes a magnet.

In an embodiment, each particle is coupled to a metal, and the magnet is configured to attract the particle to the attachment surface by attracting the metal coupled to the particle.

In an embodiment, each particle includes a metal, and the magnet is configured to attract the particle to the attachment surface by attracting the metal of the particle.

In an embodiment, each particle is coupled to a magnet, and the magnet of the attachment surface is configured to attract the particle to the attachment surface by attracting the magnet coupled to the particle.

In an embodiment, each particle includes a magnet, and the magnet of the attachment surface is configured to attract the particle to the attachment surface by attracting the magnet of the particle.

In an embodiment, the particle includes an antibody configured to attract the cancerous tissue.

In an embodiment, the antibody is coupled to a material configured to destroy the cancerous tissue at least in part by generating hyperthermia in the vicinity of the site including the cancerous tissue.

In an embodiment, the attachment surface of the filter is coupled to an antibody configured to attract at least a portion of the antibody coupled to the material configured to generate hyperthermia.

In an embodiment, the material configured to generate hyperthermia includes a metal, and the attachment surface of the filter is coupled to a chelator.

In an embodiment, the chelator is configured to attract the metal.

In an embodiment, the particles include generally toxic particles selected from, the group consisting of: chemotherapeutic particles and radiotherapeutic particles.

In an embodiment, each particle of selected particles is coupled to at least one element selected from the group consisting of: a magnet, an antibody, and a ligand, and the attachment surface is configured to capture the selected element coupled to the particle.

In an embodiment:

the selected element includes the antibody,

each particle is coupled to a respective antibody, and

the attachment surface of the filter is coupled to an antibody configured to attract the antibody coupled to the particle.

In an embodiment, the antibody coupled to the particle is coupled to a material configured to generate hyperthermia in the vicinity of the site including the cancerous tissue.

In an embodiment, the antibody coupled to the attachment surface of the filter is configured to attract at least a portion of the antibody coupled to the material configured to generate hyperthermia in the vicinity of the site including the cancerous tissue.

In an embodiment, the material configured to generate hyperthermia includes a metal, and the attachment surface of the filter is coupled to a chelator.

In an embodiment, the chelator is configured to attract the metal.

In an embodiment, the attachment surface includes an energy transducer.

In an embodiment:

the particles include generally toxic particles selected from the group consisting of: chemotherapeutic particles and radiotherapeutic particles,

each particle being coupled to a material that is sensitive to energy from the energy transducer, and

the energy transducer is configured to transmit energy configured to detoxify the selected particle at least in part by reacting with the material coupled thereto.

In an embodiment, the material is sensitive to electrical energy, and the transducer includes at least one electrode.

In an embodiment, the material includes a material that is sensitive to light, and the transducer includes a light source.

In an embodiment, the material includes a material that is sensitive to infrared radiation, and the transducer is configured to transmit infrared radiation.

In an embodiment, the material includes a material that is sensitive to ultraviolet radiation, and the transducer is configured to transmit ultraviolet radiation.

In an embodiment, the material includes a material that is sensitive to ultrasound energy, and the transducer is configured to transmit ultrasound energy.

In an embodiment:

the material includes a material that is sensitive to light, and

the apparatus further includes a catheter coupled at a distal end thereof to a light source, the light source configured to detoxify the particle at least in part by illuminating the material coupled thereto.

In an embodiment, the catheter is configured to be disposed at a site downstream of the cancerous tissue.

In an embodiment, the catheter is configured to be disposed at a site upstream of non-cancerous tissue.

In an embodiment, the energy transducer includes an ultrasound transducer.

In an embodiment, the particles include generally toxic particles selected from the group consisting of: chemotherapeutic particles and radiotherapeutic particles, each particle being coupled to a material that is sensitive to ultrasound energy, and ultrasound transmitted from the ultrasound transducer is configured to detoxify the particle at least in part by reacting with the material coupled thereto.

In an embodiment, the filter is configured to be coupled to a catheter while being advanced to the vicinity of the site.

In an embodiment, the filter is configured to remain attached to the catheter the entire time that the filter is in the vicinity of the site.

In an embodiment, the filter is configured to be separated from the catheter, while within the patient's body, after having reached the vicinity of the site.

In an embodiment, the apparatus includes an energy source in communication with the body of the patient, each particle is coupled to a material that is responsive to energy transmitted thereto from the energy source, and the particle is directed toward a vicinity within the body of the patient in response to the energy transmitted from the energy source to the material coupled to the particle.

In an embodiment, each particle is coupled to a material that is responsive to ultrasound energy applied to the material, and the energy source includes an ultrasound transducer.

In an embodiment, each particle is coupled to a material that is responsive to electrical energy, and the energy source includes at least one electrode.

In an embodiment, the apparatus is configured to direct the particle toward the filter.

In an embodiment, the apparatus is configured to direct the particle away from non-cancerous tissue.

In an embodiment, the apparatus is configured to direct the particle toward the cancerous tissue.

There is further provided, in accordance with an embodiment of the present invention, a method, including:

administering to a patient generally toxic particles selected from the group consisting of: chemotherapeutic particles, radiotherapeutic particles, radiopaque dye particles, and ultrasound contrast agent particles; and

placing a flow restricting element configured for placement into vasculature of a patient, and to reduce but not eliminate flow of the selected particles through the element.

In an embodiment, placing the flow restricting element includes placing a flow restricting element shaped to define an internal channel configured for restricting flow therethrough.

In an embodiment, placing the flow restricting element includes placing the flow restricting element in the patient for a period of less than one month.

There is still further provided, in accordance with an embodiment of the present invention, a method, including:

placing a filter in a body of a patient;

administering particles to cancerous tissue of the patient; and

capturing the particles with the filter, while the filter is in the body of the patient.

In an embodiment, administering includes administering using a technique selected from the group consisting of: intravenous administration, transcatheter administration, and oral administration.

In an embodiment, placing the filter includes placing the filter at a venous site downstream of the cancerous tissue.

In an embodiment, placing the filter includes restricting blood flow from the cancerous tissue.

In an embodiment, placing the filter includes increasing blood pressure within the cancerous tissue.

In an embodiment, placing the filter includes reducing blood circulation to the cancerous tissue.

In an embodiment, placing the filter includes effecting hypoxia-induced cell death of the cancerous tissue.

In an embodiment, placing the filter includes placing the filter in vasculature of the patient, upstream of non-cancerous tissue of the patient.

In an embodiment, placing the filter includes enhancing diffusion of the particles to the cancerous tissue.

In an embodiment, administering the particles includes inducing hyperthermia of the particles using radiofrequency energy.

In an embodiment, allowing the antibody to detoxify the particles includes allowing the antibody to change a conformation of each of the particles bound thereto.

There is yet further provided, in accordance with an embodiment of the present invention, a method, including:

placing a flow restricting element in a vicinity of cancerous tissue of a patient; and

reducing blood circulation through the cancerous tissue in response to the placing.

In an embodiment, placing the flow restricting element includes placing the flow restricting element outside of vasculature of the patient.

In an embodiment, placing the flow restricting element includes placing the flow restricting element within vasculature of the patient, upstream of the cancerous tissue.

In an embodiment, reducing the blood circulation includes effecting hypoxia-induced cell death of the cancerous tissue.

In an embodiment, placing the flow restricting element includes restricting blood circulation away from the cancerous tissue in response to the placing.

In an embodiment, placing the flow restricting element includes increasing blood pressure within the cancerous tissue in response to the restricting.

In an embodiment, the method includes administering to the patient nanoparticles, and placing the flow restricting element includes enhancing diffusion of nanoparticles to the cancerous tissue.

In an embodiment, the method includes administering to the patient particles configured to treat the cancerous tissue selected from the group consisting of: chemotherapeutic particles and radiotherapeutic particles, and placing the flow restricting element includes enhancing diffusion of particles to the cancerous tissue.

In an embodiment, the method includes administering to the patient contrast agents selected from the group consisting of: radiopaque dye particles and ultrasound contrast agent particles, and placing the flow restricting element includes enhancing diffusion of contrast agents to the cancerous tissue.

There is additionally provided, in accordance with an embodiment of the present invention, a method, including:

administering particles to cancerous tissue of a patient; and

reducing flow of the particles to non-cancerous tissue of the patient by placing a flow restricting element in a vicinity of the non-cancerous tissue.

In an embodiment, administering the particles includes administering nanoparticles.

In an embodiment, placing the flow restricting element includes placing the element in at least one blood vessel selected from the group consisting of a testicular artery, a testicular vein, an ovarian artery, and an ovarian vein.

In an embodiment, placing the flow restricting element includes placing the flow restricting element outside of vasculature of the patient.

In an embodiment, placing the flow restricting element includes placing the flow restricting element in vasculature of the patient, upstream of the non-cancerous tissue.

In an embodiment, administering the particles to the cancerous tissue includes administering antibodies configured to attract the cancerous tissue, the antibodies being coupled to a material configured to generate hyperthermia in a vicinity of the cancerous tissue, the method further includes:

generating the hyperthermia in the vicinity of the cancerous tissue by directing radiation to the material, and

destroying the cancerous tissue at least in part by the generating of the hyperthermia.

In an embodiment, administering particles to cancerous tissue of the patient includes administering to the cancerous tissue generally toxic particles selected from the group consisting of: chemotherapeutic particles, radiotherapeutic particles, radiopaque dye particles, and ultrasound contrast agent particles.

In an embodiment, placing the flow restricting element includes removing the flow restricting element following the administering of the particles.

In an embodiment, placing the flow restricting element includes replacing the flow restricting element prior to a successive administering of the particles.

In an embodiment, restricting blood circulation through the non-cancerous tissue includes inhibiting the particles from flowing through the non-cancerous tissue.

In an embodiment, the method includes:

providing antibodies configured to attract the particles,

coupling to each antibody a material configured to generate hyperthermia in a vicinity of the particles,

generating the hyperthermia in the vicinity of the particles by directing radiation toward the material, and

detoxifying the particles at least in part by the generating of the hyperthermia.

In an embodiment, directing electromagnetic radiation toward the material coupled to the particle includes illuminating a portion of a vicinity of the cancerous tissue.

In an embodiment, directing electromagnetic radiation toward the material coupled to the particle includes directing infrared radiation toward the material.

There is still additionally provided, in accordance with an embodiment of the present invention, apparatus, including:

a housing configured for placement into a body of a patient; and

a filter coupled to the housing configured to capture particles administered to the patient.

In an embodiment, the filter is configured to dwell in the patient for a period of less than one month.

In an embodiment, the particles include nanoparticles.

In an embodiment, the apparatus includes a magnetic field source in communication with the body of the patient, each particle is coupled to a material that is responsive to a magnetic field from the magnetic field source, and the particle is directed toward a vicinity within the body of the patient in response to the magnetic field from the magnetic field source.

In an embodiment, the particles include contrast agents selected from the group consisting of: radiopaque dye particles and ultrasound contrast agent particles.

In an embodiment, the filter includes at least one attachment surface configured to capture particles administered to the patient.

In an embodiment, each particle of the selected contrast agent is coupled to a ligand, and the attachment surface includes receptors possessing affinity to the ligand.

In an embodiment, the filter includes a housing, and the attachment surface includes a plurality of attachment surfaces, coupled at respective sites to the housing.

In an embodiment, the attachment surface, is coupled to at least one enzyme configured to metabolize the particles.

In an embodiment, the attachment surface is configured to be electrically charged by a first charge, and each particle is electrically charged by a second charge, the first charge configured to attract the second charge.

In an embodiment, the apparatus includes a nanocontainer configured to insulate the particles.

In an embodiment, the apparatus includes at least one electrode in communication with the attachment surface, the at least one electrode configured to apply the first charge to the attachment surface.

In an embodiment, the attachment surface includes a magnet.

In an embodiment, each particle is coupled to a metal, and the magnet is configured to attract the particle to the attachment surface by attracting the metal coupled to the particle.

In an embodiment, each particle includes a metal, and the magnet is configured to attract the particle to the attachment surface by attracting the metal of the particle.

In an embodiment, each particle is coupled to a magnet, and the magnet of the attachment surface is configured to attract the particle to the attachment surface by attracting the magnet coupled to the particle.

In an embodiment, each particle includes a magnet, and the magnet of the attachment surface is configured to attract the particle to the attachment surface by attracting the magnet of the particle.

In an embodiment, the attachment surface includes at least one type of antibody.

In an embodiment, the antibody is configured to detoxify the particles by neutralizing an active site of the particle.

In an embodiment, the antibody is configured to:

reversibly couple each particle,

detoxify the particle, and

release the particle subsequently to the detoxification of the particle.

In an embodiment, the attachment surface includes an energy transducer.

In an embodiment, each particle is coupled to a material that is sensitive to ultrasound energy, and the energy transducer is configured to transmit ultrasound energy configured to detoxify the particle at least in part by reacting with the material coupled thereto.

In an embodiment, each particle is coupled to a material that is sensitive to radiofrequency energy, and the energy transducer is configured to transmit radiofrequency energy configured to detoxify the particle at least in part by reacting with the material coupled thereto.

In an embodiment, each particle is coupled to a material that is sensitive to electromagnetic radiation, and the energy transducer is configured to transmit electromagnetic radiation configured to detoxify the particle at least in part by reacting with the material coupled thereto.

In an embodiment, the material includes a material that is sensitive to infrared radiation, and the transducer is configured to transmit infrared radiation.

In an embodiment, the material includes a material that is sensitive to light, and the transducer includes a light source.

In an embodiment, the material includes a material that is sensitive to ultraviolet radiation, and the transducer is configured to transmit ultraviolet radiation.

In an embodiment:

the material includes a material that is sensitive to light,

the apparatus further includes a catheter coupled at a distal end thereof to a light source configured detoxify the particle at least in part by illuminating the material coupled thereto,

the catheter being configured to be disposed at a site downstream of a site of administration of the particle to the patient.

In an embodiment, the apparatus includes a source of energy in communication with the body of the patient, each particle is coupled to a material that is responsive to energy transmitted thereto from the energy source, and the particle is directed toward a vicinity within the body of the patient in response to the energy transmitted from the energy source to the material coupled to the particle.

In an embodiment, each particle is coupled to a material that is responsive to ultrasound energy, and the energy source includes an ultrasound transducer.

In an embodiment, each particle is coupled to a material that is responsive to electrical energy, and the energy source includes at least one electrode.

In an embodiment, the apparatus is configured to direct the particle toward the filter.

In an embodiment, the apparatus is configured to direct the particle away from non-cancerous tissue.

In an embodiment, the apparatus is configured to direct the particle toward the cancerous tissue.

There is yet additionally provided, in accordance with an embodiment of the present invention, a method, including:

placing a filter in a body of a patient;

administering particles to vasculature of the patient; and

capturing the particles with the filter, while the filter is in the body of the patient.

In an embodiment, administering includes administering the contrast agent using a technique selected from the group consisting of: intravenous administration and transcatheter administration.

In an embodiment, the method includes visualizing a site in the body of the patient using the contrast agent, and placing the filter includes placing the filter in the vasculature downstream of the site.

In an embodiment, placing the filter includes placing the filter at site in the vasculature upstream of an organ, and capturing the contrast agent includes restricting passage of the contrast agent to a vicinity of the organ.

There is also provided, in accordance with an embodiment of the present invention, apparatus, including:

an energy transducer configured for placement in communication with a body of a patient; and

a particle configured to be administered to the patient to facilitate a medical procedure, and to be detoxified at least in part by energy transmitted from the energy transducer following the facilitation by the particle of the medical procedure.

In an embodiment, the energy transducer is configured to be disposed outside the body of the patent and to transmit energy to the administered particle.

In an embodiment, the particle is configured to be administered intravenously.

In an embodiment, the particle is configured to be administered transcatheterally.

In an embodiment, the particle is configured to be administered orally.

In an embodiment, the particle is sensitive to ultrasound energy, and the energy transducer is configured to transmit ultrasound energy configured to detoxify the particle at least in part.

In an embodiment, the particle is coupled to a material that is sensitive to ultrasound energy, and the energy transducer is configured to transmit ultrasound energy configured to detoxify the particle at least in part by reacting with the material coupled to the particle.

In an embodiment, the particle is coupled to a material that is sensitive to radiofrequency energy, and the energy transducer is configured to transmit radiofrequency energy configured to detoxify the particle at least in part by reacting with the material coupled to the particle.

In an embodiment, the particle is coupled to a material that is sensitive to radiofrequency energy, and the energy transducer is configured to transmit radiofrequency energy configured to detoxify the particle at least in part by reacting with the material coupled to the particle.

In an embodiment, the particle is configured to be coupled to a material that is sensitive to electromagnetic radiation, and the energy transducer is configured to transmit electromagnetic radiation configured to detoxify the particle at least in part by reacting with the material coupled to the particle.

In an embodiment, the particle is configured to be coupled to a material that is sensitive to at least one form of radiation selected from the group consisting of: infrared radiation and ultraviolet radiation, and wherein the energy transducer is configured to transmit the selected radiation configured to detoxify the particle at least in part by reacting with the material coupled to the particle.

In an embodiment, the particle includes a contrast agent.

In an embodiment, the contrast agent includes radiopaque dye particles.

In an embodiment, the contrast agent includes ultrasound contrast agent particles.

In an embodiment, the particle is configured to treat cancerous tissue.

In an embodiment, the particle includes a chemotherapeutic particle.

In an embodiment, the particle includes a radiotherapeutic particle.

In an embodiment, the energy transducer is configured to be disposed within the body of the patient.

In an embodiment, the energy transducer is configured to be implanted within the body of the patient.

In an embodiment, the apparatus includes a catheter having a distal end, the energy transducer is configured to be coupled to the catheter at the distal end thereof, and to be advanced transcatheterally to a site within the body of the patient following the administration of the particle.

In an embodiment:

the particle is configured to treat cancerous tissue,

the catheter is configured to be disposed at a site downstream of the cancerous tissue, and

the energy transducer is configured to transmit energy sufficiently to detoxify the particle once the particle is downstream of the cancerous tissue.

In an embodiment, the catheter is configured to be disposed at a site upstream of a non-cancerous tissue.

There is also provided, in accordance with an embodiment of the present invention, a method, including:

placing an energy transducer in communication with a body of a patient;

administering to the patient a particle configured to facilitate a medical procedure and to be detoxified at least in part by energy transmitted from the energy transducer; and

detoxifying the particle by directing energy toward the particle.

In an embodiment, administering the particle includes treating tissue of the patient, and placing the energy transducer includes placing the energy transducer downstream of the tissue.

In an embodiment, placing the energy transducer includes restricting passage of the particle toward tissue of the patient, and placing the energy transducer includes placing the energy transducer upstream of the tissue.

The present invention will be more fully understood from the following detailed description of embodiments thereof, taken together with the drawings, in which:

### BRIEF DESCRIPTION OF THE DRAWINGS

FIG. 1 is a schematic illustration of a filter housing comprising attachment surfaces, in accordance with an embodiment of the present invention;

FIG. 2 is a schematic illustration of a lengthwise cross-section of the filter of FIG. 1, in accordance with an embodiment of the present invention;

FIG. 3 is a schematic illustration of an axial cross section of a flow restricting element, in accordance with an embodiment of the present invention;

FIG. 4 is a schematic illustration of the flow restricting element of FIG. 3, in accordance with an embodiment of the present invention;

FIG. 5 is a schematic illustration of the flow restricting element, in accordance with another embodiment of the present invention;

FIG. 6 is a schematic illustration of the filter housing of FIG. 1, in accordance with an embodiment of the present invention;

FIG. 7 is a schematic illustration of the flow restricting element, in accordance with an embodiment of the present invention;

FIG. 8 is a schematic illustration of a longitudinal cross-section of the filter of FIG. 1 comprising a light source, in accordance with an embodiment of the present invention; and

FIG. 9 is a schematic illustration of a catheter, in accordance with an embodiment of the present invention.

### DETAILED DESCRIPTION OF THE EMBODIMENTS

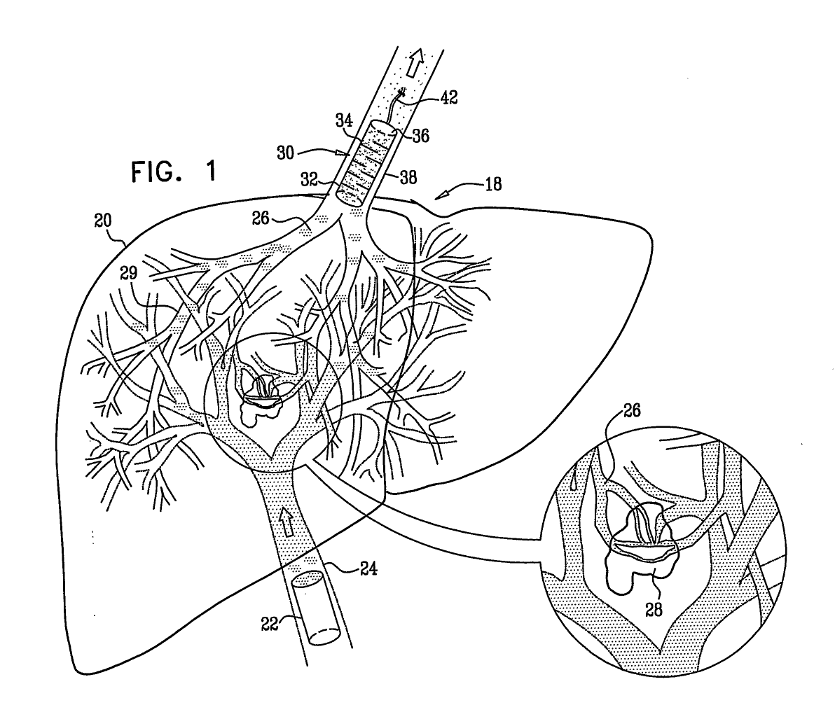

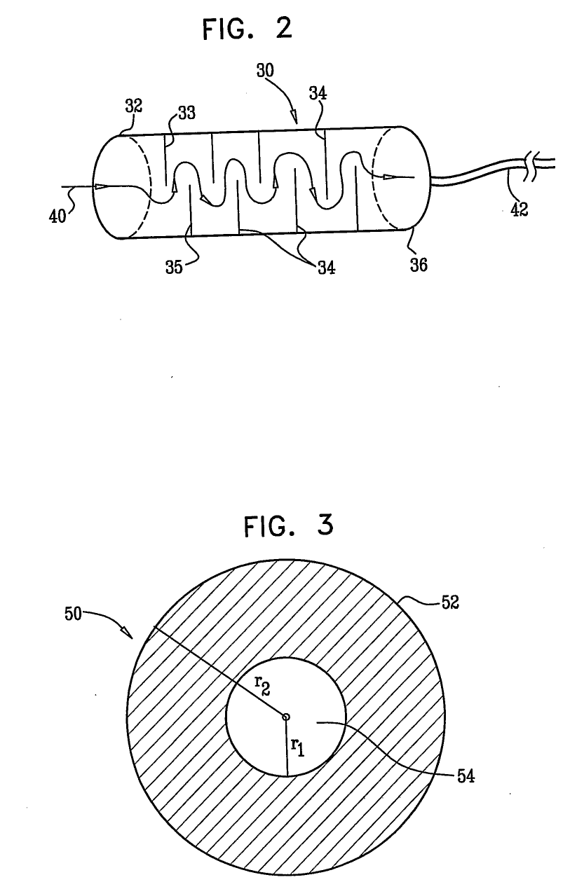

Reference is now made to FIGS. 1, and 2, which are schematic illustrations of apparatus 18, comprising a filter 30 that comprises one or more attachment surfaces 34, in accordance with an embodiment of the present invention. FIG. 1 shows filter 30 at a venous site of the vasculature of a patient, downstream of an organ 20 containing cancerous tissue 28. FIG. 2 shows a lengthwise cross-section of filter 30.

As shown in FIG. 1, organ 20 is the liver by way of example, and particles 26 are emitted from a source 22 positioned in hepatic artery 24, by way of example, located upstream of organ 20. Typically, the particles comprise generally toxic treatment particles (e.g., chemotherapeutic particles and/or radiotherapeutic particles) and/or generally toxic contrast agent particles (e.g., radiopaque dye particles, and/or ultrasound contrast agent particles. Typically, particles 26 comprise nanoparticles. In an embodiment, source 22 comprises a drug-delivery catheter. Alternatively, source 22 comprises an implanted drug-delivery pump. While a substantial portion of particles 26 which are administered by source 22 are typically uptaken by cancerous site 28, some particles 26 escape, continuing along hepatic vasculature 29 of the patient. Placing filter 30 downstream of cancerous site 28 in hepatic vein 38, by way of example, allows for excess particles 26, potentially toxic to healthy tissue, to be absorbed via attachment surfaces 34 of filter 30. Particles 26 contained in the bloodstream flow through a proximal end 32 of filter 30, and are attracted to and/or captured by attachment surfaces 34. This allows filtered blood to pass through a distal end 36 of filter 30, with fewer (or substantially devoid of) particles 26.

Attachment surfaces 34 of filter 30 typically comprise means for attracting the treatment particles and/or contrast agent particles. For example, a magnet may be used to attract magnetically-sensitive particles bound to the treatment particles and/or to the contrast agent particles. Alternatively, antibodies functioning as receptors possessing affinity to ligands associated with the treatment particles and/or contrast agent particles are used to attract the treatment particles and/or contrast agent particles.

In some embodiments, a protein is coupled to each particle 26, and the antibodies coupled to the attachment surfaces are configured to bind to the protein coupled to the particle. In some embodiments, the particle remains bound to attachment surface 34 of filter 30 via the bond between the antibody and the protein coupled to particle 26. Alternatively or additionally, the antibody is configured to neutralize and/or detoxify particle 26 bound thereto either directly or indirectly (i.e., via the protein coupled to the particle). In such an embodiment, once directly or indirectly bound to particle 26, the antibody undergoes a conformational change in order to physically reduce the effectiveness of particle 26. In either embodiment, following the detoxification of particle 26, the particle either remains coupled to filter 30 and/or is allowed to migrate therefrom.

In some embodiments, attachment surfaces 34 are coupled to enzymes whose active sites target and detoxify particles 26. For embodiments in which chemotherapy particles are administered to the patient, the enzyme typically comprises an enzyme (e.g., aldehyde dehydrogenase or glutathion-S-transferase) that metabolizes and detoxifies chemicals of the chemotherapy particle.

In some embodiments of the present invention, attachment surfaces 34 of filter 26 are charged in response to an application of voltage thereto. In some embodiments, the voltage is applied to surfaces 34 using an electrode (not shown) implanted within the body of the patient adjacently to filter 30. In such an embodiment, particles 26 comprise charged nanoparticles which are attracted to the charged attachment surfaces of filter 30. The charged nanoparticles are typically insulated by and enveloped within nanocontainers, e.g., buckyballs, when administered to the patient.

For some applications, placing filter 30 downstream of organ 20 restricts blood flow 40 away from organ 20, effecting an increase in blood pressure within hepatic vasculature 29. Consequently, diffusion of particles 26 to cancerous site 28 is enhanced.

In an embodiment, placing filter 30 downstream of cancerous site 28 restricts circulation to cancerous site 28, inhibiting activity or effecting hypoxia-induced cell death in cancerous site 28, independently or in combination with administering particles 26.

Filter 30 is typically configured to be coupled to a catheter 42 while being advanced to the site of cancerous tissue 28, and, in some embodiments, catheter 42 is configured to remain attached to filter 30 the entire time that filter 30 is in the vicinity of the site. Alternatively, catheter 42 is configured to be separated from filter 30 after having reached the vicinity of the site.

In an embodiment, particles 26 are heated in situ by the application of radiofrequency energy, e.g., using techniques known in the art and/or described in references cited in the Background section of the present patent application.

FIG. 2 shows the interior of filter 30 illustrated in lengthwise cross-section. Blood flow 40 containing particles 26 enters proximal end 32 of filter 30. As shown in FIG. 2, attachment surfaces 34 are disposed with respect to the housing of filter 30 such that a first one of the surfaces 33 attracts and captures a first subset of particles 26 within the blood flow 40 flowing therethrough, and a second one of the surfaces 35 attracts and captures a second subset of particles 26. Consequently, the concentration of particles 26 within blood flow 40 gradually decreases as subsequent attachment surfaces 34 capture particles 26. Attachment surfaces 34 are configured such that ultimately, a reduced number of particles 26 pass through distal end 36 to continue through vasculature of the patient. In an embodiment, antibodies to the cancerous tissue are coupled to materials, e.g., gold, that are configured to generate hyperthermic conditions in the cancerous tissue in response to applied stimulation. Typically, antibodies are administered to the patient, and bind to the cancerous tissue. Subsequently, a source of radiation, e.g., a transmitter disposed outside the body of the patient, transmits radiation, e.g., radiofrequency energy, toward the vicinity of the cancerous tissue in order to heat the materials bound to the antibodies. In response to the heating of the materials coupled to the antibodies that are now bound to the cancerous tissue, hyperthermic conditions are generated which destroy the cancerous tissue. Antibodies that did not bind to the cancerous tissue are removed from the blood stream by filter 30. In an embodiment, attachment surfaces 34 of filter 30 are coupled to antibodies which attract at least a portion of the antibodies bound to the hyperthermia-inducing materials. Alternatively, attachment surfaces 34 of filter 30 are coupled to chelators which attract the hyperthermia-inducing materials.

The antibodies coupled to the hyperthermia-inducing materials may be administered to the patient in combination with or independently of the chemotherapeutic particles.

It is to be appreciated that the arrangement of attachment surfaces 34 shown in FIG. 2 is by way of illustration and not limitation, and that the scope of the present invention includes surfaces aligned in parallel to capture particles 26 (rather than in series, as shown). Similarly, the scope of the present invention includes the use of a single attachment surface 34, typically configured to provide a relatively large attachment surface area to which particles 26 may bind. In an embodiment, filter 30 is generally shaped like a standard stent, and attachment surface 34 is formed as a coating on exposed surfaces of the stent.

Reference is now made to FIG. 3, which is a schematic illustration of an axial cross section of apparatus 50 comprising a flow restricting element 52 shaped to define a channel such as an internal channel 54, in accordance with an embodiment of the present invention. Internal channel 54 of flow restricting element 52 is shaped to restrict blood flow 40 therethrough. A radius r2 of flow restricting element 52 and a radius r1 of internal channel 54 are typically selected such that r1/r2 is between about 0.50 and 0.95, thereby reducing the area for blood to flow through channel 54. For some applications, other values of r1/r2 are used, outside of this range. In an embodiment, flow restricting element 52 is generally shaped like a standard stent.

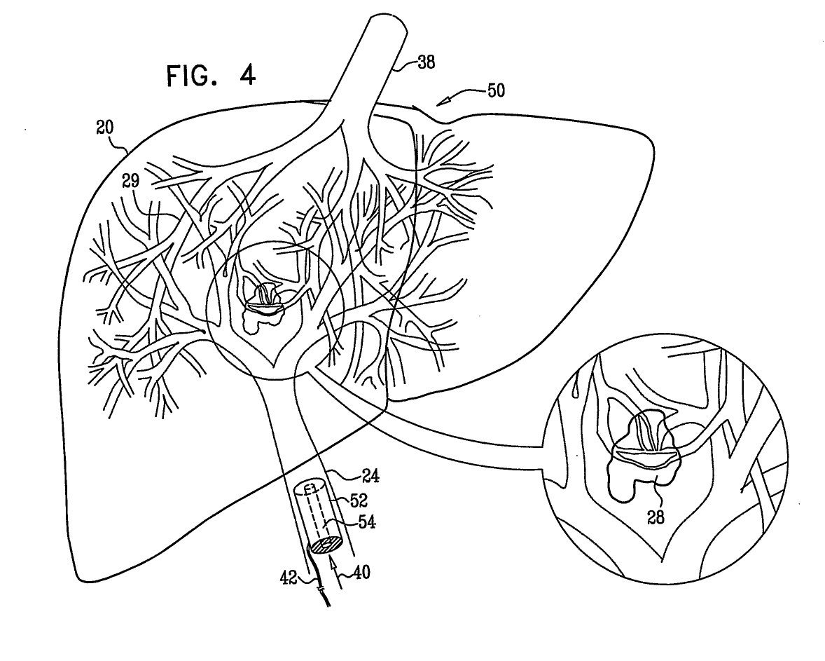

Reference is now made to FIG. 4, which is a schematic illustration of flow restricting element 52 placed at a site within patient vasculature upstream of cancerous tissue 28, in accordance with an embodiment of the present invention. Flow restricting element 52 is configured to be coupled to catheter 42 while being advanced to the site of cancerous tissue 28. In some embodiments, catheter 42 is configured to remain attached to flow restricting element 52 the entire time that flow restricting element 52 is in the vicinity of the site. Alternatively, catheter 42 is configured to be separated from flow restricting element 52 after having reached the vicinity of the site. In either case, blood flow 40 to cancerous tissue 28 is restricted by internal channel 54 of flow restricting element 52. Flow restricting element 52 is placed in the vicinity of cancerous site 28 in combination with or in the absence of particles 26.

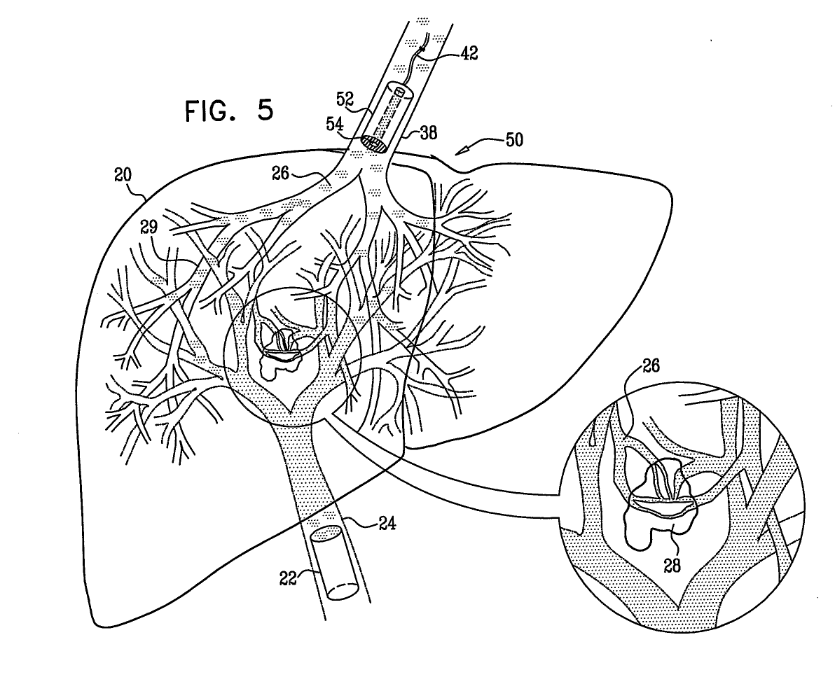

Reference is now made to FIG. 5, which is a schematic illustration of flow restricting element 52 placed at a site within patient vasculature downstream of cancerous tissue 28, in accordance with an embodiment of the present invention. As a supplement to particles 26 administered in a cancer-treatment or imaging procedure, flow restricting element 52 is positioned by catheter 42 within vein 38. Blood flow 40 downstream of cancerous tissue 28 is restricted by internal channel 54 of flow restricting element 52, thereby increasing blood pressure within vasculature 29. Consequently, diffusion of particles 26, from source 22, through cancerous tissue 28 is enhanced.

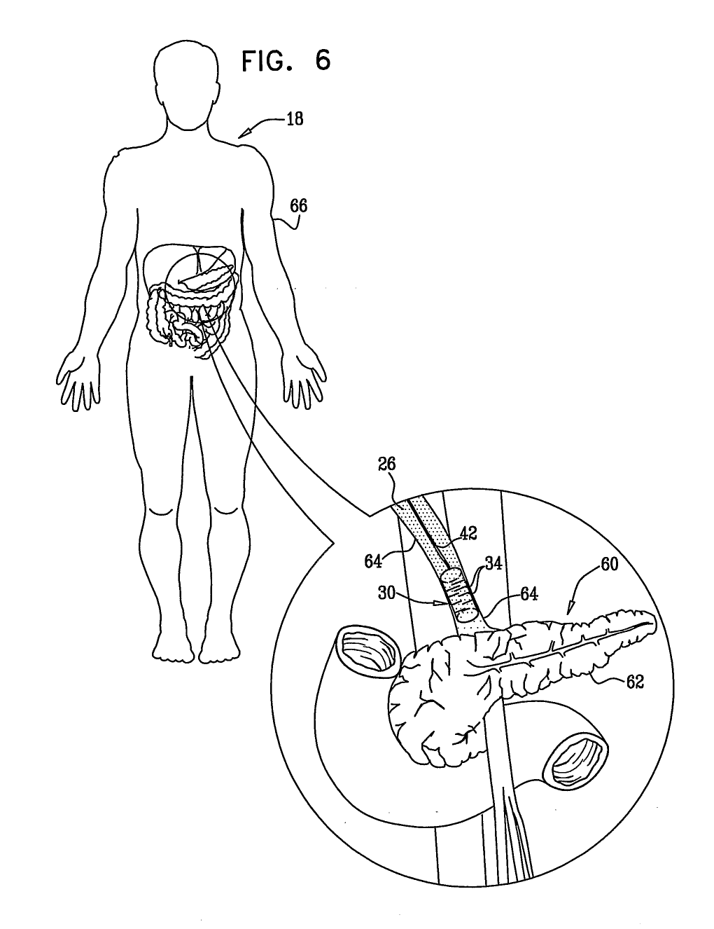

Reference is now made to FIG. 6, which is a schematic illustration of apparatus 18 comprising filter 30 placed at a site within vasculature of a patient 66, upstream of non-cancerous tissue 60, in accordance with an embodiment of the present invention. Non-cancerous tissue 60 is a pancreas 62, by way of example, and filter 30 is placed by catheter 42 in an artery 64 such as a pancreatic artery. Alternatively, artery 64 is a testicular artery or an ovarian artery, and filter 30 reduces the likelihood or duration of infertility secondary to chemotherapy. Excess particles 26, escaping from a cancerous site to which they have been administered (e.g., by transcatheter, intravenous, or oral administration), are harmful and potentially toxic to non-cancerous tissue 60. Placing filter 30 in artery 64, upstream of non-cancerous tissue 60, allows for excess particles 26 to be absorbed by attachment surfaces 34 of filter 30, reducing exposure of non-cancerous tissue 60 to particles 26.

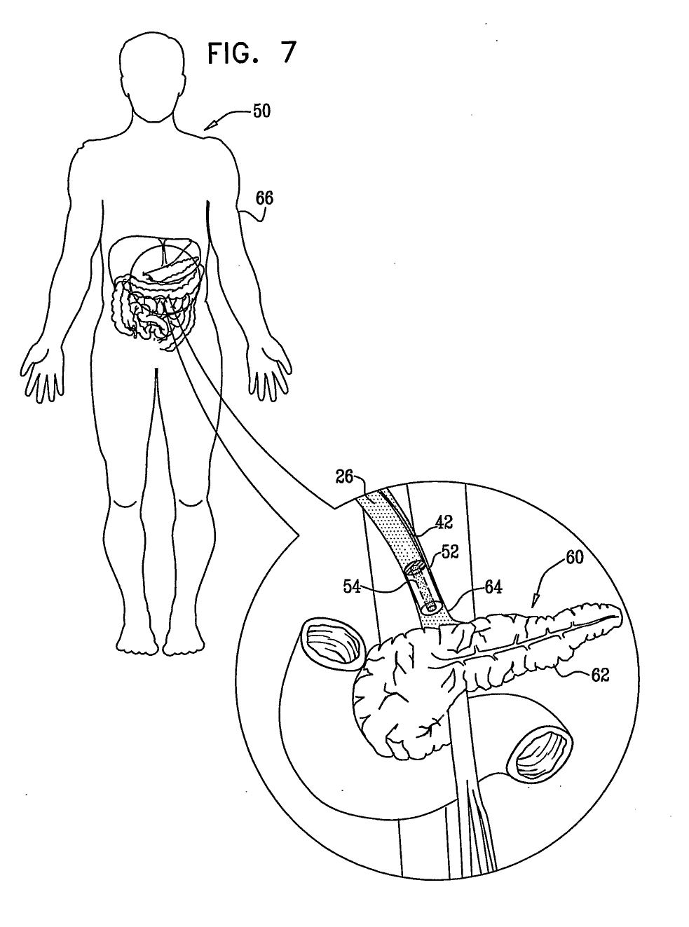

Reference is now made to FIG. 7, which is a schematic illustration of flow restricting element 52, placed at a site within vasculature of patient 66, upstream of non-cancerous tissue 60, in accordance with an embodiment of the present invention. Non-cancerous tissue 60 is a pancreas 62, by way of example, and flow restricting element 52 is placed by catheter 42 in pancreatic artery 64. Alternatively, artery 64 is a testicular artery or an ovarian artery, and flow restricting element 52 reduces the likelihood or duration of infertility secondary to chemotherapy. The narrow diameter of internal channel 54 of flow restricting element 52 transiently restricts blood flow to non-cancerous tissue 60 during the administration of particles 26 to a cancerous site within patient 66, thereby reducing exposure of non-cancerous tissue 60 to particles which may escape the cancerous site. As appropriate, particles 26 may be administered by catheter, intravenously, or orally.

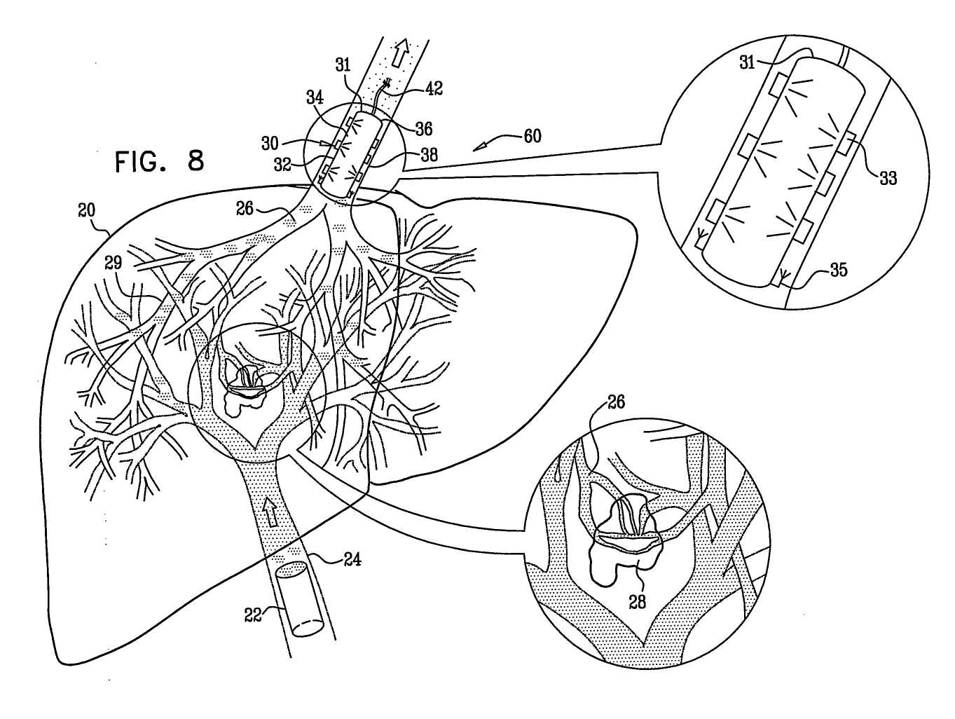

Reference is now made to FIG. 8, which is a schematic longitudinal cross-sectional illustration of apparatus 60 comprising filter 30 comprising a source of radiation 31 of apparatus 60, in accordance with an embodiment of the present invention. Typically, radiation source 31 comprises a plurality of energy transducers 33 which are disposed along an inner wall of filter 30. Typically, the inner wall of filter 30 is shaped to define a lumen of filter 30 for passage therethrough of the blood of the patient. As shown, transducers 33 comprise light emitting diodes (LED). In such an embodiment, particles 26 are coupled to a light-sensitive material. Light is transmitted from the LEDs of radiation source 31 into the lumen of filter 30, and reacts with the light-sensitive material coupled to particles 26. The transmitted light indirectly detoxifies particles 26 by reacting with the light-sensitive material coupled thereto. It is to be noted that particles 26 described herein are coupled to a light-sensitive material by way of illustration and not limitation. For example, particles 26 may be coupled to a material that is sensitive to any type of electromagnetic radiation, e.g., infrared radiation, visible light, and/or ultraviolet radiation.

In some embodiments, particles 26 are coupled to materials which are sensitive to radiofrequency energy or ultrasound energy. For example, particles 26 may be coupled to a material that is sensitive to ultrasound energy. In such an embodiment, energy transducers 33 of radiation source 31 comprise ultrasound transducers. Typically, the ultrasound transducers of radiation source 31 receive ultrasound energy transmitted from a source external to the body of the patient. Responsively, the ultrasound transducers direct the ultrasound energy toward particles 26 passing through filter 30. The ultrasound energy reacts with the ultrasound-sensitive materials coupled to particles 26 in order to detoxify particles 26.

In an embodiment, radiation source 31 of filter 30 comprises a receiver 35 which receives radiated energy transmitted from a source of radiation which is disposed outside the body of the patient. The energy received by receiver 35 is then directed to and applied within the lumen of filter 30. In some embodiments, the energy transmitted from outside the body of the patient is configured to actuate energy transducers 33. For example, for embodiments in which transducers 33 comprise LEDs, the energy transmitted from the source of radiation disposed outside the body of the patient may be used to actuate the LEDs to transmit light within the lumen of filter 30.

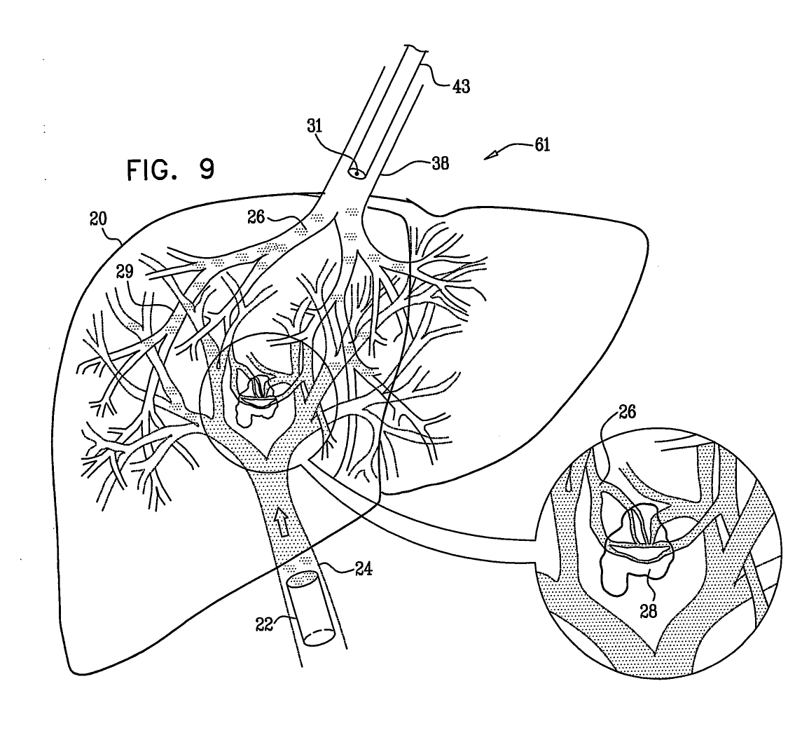

Reference is now made to FIG. 9, which is a schematic illustration of apparatus 62 comprising a catheter 43 disposed downstream of the cancerous tissue, in accordance with an embodiment of the present invention. Catheter 43 includes radiation source 31 disposed on its distal end. In some embodiments, radiation source 31 comprises a light source, e.g., a light emitting diode (LED) or an optical fiber. In such an embodiment, particles 26 are coupled to a light-sensitive material which reacts with the light transmitted from radiation source 31 in order to detoxify particles 26.

For embodiments in which particles 26 are coupled to materials that are sensitive to radiofrequency energy or ultrasound energy, radiation source 31 comprises a suitable transducer. The materials coupled to particle 26 react with the energy emitted from the transducer, and particles 26 are detoxified.

Techniques described hereinabove may be used in combination with a medical procedures in which a contrast agent, e.g., a radiopaque dye or an ultrasound contrast agent, is administered, e.g., orally, transcatheterally, or intravenously. For example, a radiopaque dye may be administered to the patient during a diagnostic procedure, e.g., a procedure for locating a position of an aneurysm or a procedure for locating a position of a thrombosis. In such an embodiment, a filter is positioned downstream of the aneurysm in order to remove the radiopaque dye from the bloodstream of the patient. Alternatively or additionally, the filter is placed upstream of an organ so as to restrict passage of the radiopaque dye into a vicinity of the organ. In another embodiment, a radiopaque dye is administered during a therapeutic procedure, e.g., implantation of a coronary stent, and a filter is positioned to remove the dye from the bloodstream and reduce any toxicity associated with the dye.

In some embodiments, the contrast agent is coupled to a material that is sensitive to energy, e.g., radiofrequency energy, ultrasound energy, or electromagnetic energy. In such an embodiment, an energy transducer is disposed in communication with the body of the patient. For example, the energy transducer may be disposed at an external surface of the body or introduced transcatheterally within the body. For example, the contrast agent may be coupled to a material that is sensitive to ultrasound. For embodiments in which the contrast agent is coupled to a material that is sensitive to ultrasound, the energy transducer comprises an ultrasound transducer.

Reference is now made to FIGS. 1-9. It is to be noted that apparatus described herein for detoxifying toxic particles 26 administered to the body of the patient may be used in combination with apparatus configured to direct particles 26 toward the apparatus for detoxifying the toxic particles. In some embodiments, particles 26 are coupled to a material that is positively responsive to transmitted energy (e.g., ultrasound energy, radiofrequency energy, and/or electromagnetic energy). In some embodiments, the energy is transmitted from a source external to the body of the patient. Alternatively or additionally, the energy is transmitted from a source within the body of the patient. In response to the energy applied to the material, the particle is deflected toward a vicinity of choice, e.g., toward filter 30. For example, each particle 26 may be coupled to a material that is responsive to ultrasound energy. In response to ultrasound energy transmitted from an ultrasound transducer, particles 26 are deflected toward filter 30.

Alternatively or additionally, particles 26 are coupled to and/or comprise magnetic and/or metallic particles. In response to magnetic fields applied to the body of the patient, particles 26 are deflected toward filter 30. In some embodiments, particles 26 comprise radiotherapeutic nanoparticles which are synthesized such that they generate magnetic fields. Alternatively, these particles are synthesized such that they respond to magnetic fields applied thereto.

For some applications, each particle 26 is coupled to a material that is responsive to electromagnetic energy, e.g., ultraviolet energy, visible light, or infrared energy, and is detoxified in response to the applied energy. For some applications, each particle 26 is coupled to a material which is responsive to electrical energy. In such an embodiment, electrodes are positioned in communication with the body of the patient and are used to deflect particles 26 within the vasculature of the patient via the materials coupled to particles 26.

In like manner, applying energy toward the materials positively responsive to transmitted energy facilitates deflection of particles 26 away from tissue not designated for treatment and/or diagnosis. For example, chemotherapeutic nanoparticles used to treat cancerous tissue may be coupled to the above-mentioned materials. In response to energy transmitted toward the material, the chemotherapeutic nanoparticles may be deflected away from non-cancerous tissue. Alternatively or additionally, particles 26 may be deflected toward the cancerous tissue.

It is to be noted that the scope of the present invention includes the use of particles, e.g., nanoparticles, which are synthesized such that they are susceptible at at least a portion thereof to energy applied thereto. When energy is transmitted toward the particle, the particles are detoxified because the energy weakens their construction. Alternatively or additionally, the particles described herein may be synthesized such that they are susceptible at least a portion thereof to chemicals applied thereto. When the chemicals are applied to the particles, the particles are detoxified because the applied chemicals interfere with chemical bonds of the particle.

It is to be noted that the energy transducer described herein may be used independently of or in combination with filter 30 described herein.

Although devices and methods are shown in some figures as being used near the liver or pancreas, it is to be understood that the methods and devices described herein can be used with respect to other sites within a patient's body, as well. Similarly, the scope of the present invention includes using a filter to capture particles other than chemotherapeutic particles and radiopaque dye particles, such as radioimmunotherapy particles, and any drug particles not intended to treat cancer. Thus, the scope of the present invention includes uses of the filter and flow restricting element other than those described hereinabove and shown in the figures. For example, the invention may be applied to treatments other than chemotherapy, or to alternative forms of chemotherapy. Furthermore, the scope of the present invention is not restricted to use as shown in FIGS. 1-9, and may instead be used in the treatment or imaging of cancer or other anatomy or pathology elsewhere in the body. Lastly, the scope of the present invention includes filters having shapes and configurations of the attachment surfaces other than those shown in the figures. For example, the attachment surfaces may be embodied in a tight mesh, through which blood of the patient passes.

It is to be appreciated that all embodiments described herein with respect to transcatheter administration of cancer-treatment particles and contrast agent particles may be practiced as well using other administration techniques, such as intravenous or oral administration.

It is to be understood that although some embodiments are described herein with respect to a property of a material coupled to a particle, the scope of the present invention includes incorporating such a property into the particle itself.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising:
a filter configured for placement into a body of a patient, in a vicinity which is downstream of a site including cancerous tissue, the filter comprising;
an attachment surface configured to capture particles administered to treat the tissue; and
a flow restricting element configured to restrict blood flow coming from the tissue and increase blood pressure within the tissue.

2-54. (canceled)

55. A method, comprising:
placing a filter in a body of a patient;
administering particles to cancerous tissue of the patient; and
capturing the particles with the filter, while the filter is in the body of the patient.

56-58. (canceled)

59. The method according to claim 55, wherein placing the filter comprises placing the filter at a venous site downstream of the cancerous tissue.

60. The method according to claim 55, wherein placing the filter comprises restricting blood flow coming from the cancerous tissue.

61. The method according to claim 55, wherein placing the filter comprises increasing blood pressure within the cancerous tissue.

62-63. (canceled)

64. The method according to claim 55, wherein placing the filter comprises placing the filter in vasculature of the patient, upstream of non-cancerous tissue of the patient.

65. (canceled)

66. The method according to claim 55, wherein administering particles comprises administering to the tissue of the patient generally toxic particles selected from the group consisting of: chemotherapeutic particles, radiotherapeutic particles, radiopaque dye particle, and ultrasound contrast agent particles.

67-69. (canceled)

70. The method according to claim 66, wherein placing the filter comprises enhancing diffusion of the particles to the cancerous tissue.

71. The method according to claim 66, wherein placing the filter comprises removing the filter following the administering of the particles.

72. The method according to claim 66, wherein placing the filter comprises replacing the filter prior to a successive administering of the particles.

73-78. (canceled)

79. The method according to claim 66, wherein placing the filter comprises placing a filter coupled to an antibody within the body of the patient, and wherein capturing the particles comprises allowing the antibody to bind to the particles.

80. The method according to claim 79, wherein allowing the antibody to bind to the at least a portion of the particles comprises allowing the antibody to detoxify the particles coupled thereto.

81-82. (canceled)

83. The method according to claim 80, further comprising allowing the particles to be released from the filter following the detoxification of the particles by the antibodies.

84-89. (canceled)

90. A method, comprising:
placing a flow restricting element in a vicinity of cancerous tissue of a patient; and
reducing blood circulation through the cancerous tissue in response to the placing.

91. The method according to claim 90, wherein placing the flow restricting element comprises placing the flow restricting element outside of vasculature of the patient.

92-93. (canceled)

94. The method according to claim 90, wherein placing the flow restricting element comprises restricting blood circulation away from the cancerous tissue in response to the placing.

95. The method according to claim 90, wherein placing the flow restricting element comprises increasing blood pressure within the cancerous tissue in response to the restricting.

96. (canceled)

97. The method according to claim 90, further comprising administering to the patient particles configured to treat the cancerous tissue selected from the group consisting of: chemotherapeutic particles and radiotherapeutic particles, and wherein placing the flow restricting element comprises enhancing diffusion of particles to the cancerous tissue.

98. The method according to claim 90, further comprising administering to the patient contrast agents selected from the group consisting of: radiopaque dye particles and ultrasound contrast agent particles, and wherein placing the flow restricting element comprises enhancing diffusion of contrast agents to the cancerous tissue.

99-207. (canceled)

208. The method according to claim 1, wherein the particles include generally toxic particles selected from the group consisting of: chemotherapeutic particles and radiotherapeutic particles, and wherein the flow restricting element is configured to enhance diffusion of the selected particles into the cancerous tissue by increasing the blood pressure within the tissue.
